(19) United States
(12) Patent Application Publication
(10) Pub. No.: US 2026/0083572 A1
(43) Pub. Date: Mar. 26, 2026

(54) EXPANDABLE INTERVERTEBRAL IMPLANT, BONE GRAFT DELIVERY, AND METHODS FOR USING SAME

(71) Applicant: Spinal Surgical Strategies, Inc., d/b/a Kleiner Device Labs, Incline Village, NV (US)

(72) Inventors: Jeffrey B. Kleiner, Denver, CO (US); Alan Burkholder, Denver, CO (US); Gregory Causey, Erie, CO (US); Edward Grimberg, JR., Golden, CO (US)

(73) Assignee: Spinal Surgical Strategies, Inc., d/b/a Kleiner Device Labs, Incline Village, NV (US)

(21) Appl. No.: 19/302,263

(22) Filed: Aug. 18, 2025

Related U.S. Application Data
(60) Provisional application No. 63/698,439, filed on Sep. 24, 2024.
(60) Provisional application No. 63/698,887, filed on Sep. 25, 2024.

(51) Int. Cl. A61F 2/44; A61F 2/30; A61F 2/46

(52) U.S. Cl. CPC A61F 2/4425; A61F 2002/30326; A61F 2002/30405; A61F 2002/30518; A61F 2002/443; A61F 2/4601

(57) ABSTRACT

An implant for being disposed in a spine of a patient, and a method of using same, has an upper plate, a lower plate, and a wedge piece disposed between the upper plate and the lower plate. The implant can have a driver for moving the implant to an expanded condition, and surfaces for driving movement of the implant, and surfaces for directing material through the implant.

### CROSS-REFERENCE TO RELATED APPLICATIONS

This application claims the benefit of provisional application Ser. No. 63/698,439, filed Sep. 24, 2024 and application Ser. No. 63/698,887, filed Sep. 25, 2024, the disclosures of which are incorporated by reference herein in their entirety.

### TECHNICAL FIELD

This disclosure relates to orthopedic surgery and more specifically to an apparatus and method for placement of an expandable spinal implant in a patient's spine.

### BACKGROUND

Individuals who suffer degenerative disc disease, natural spine deformations, a herniated disc, spine injuries, or other spine disorders, may require surgery on the affected region of the spine in order to address the clinical situation. Among the various surgical interventions, one procedure calls for an implant to be inserted in between the patient's adjacent vertebrae to address the patient's specific needs. Such procedures vary depending on the nature and extent of the injury or deformity. For example, there are multiple types of lumbar procedures, including posterior lumbar interbody fusion (“PLIF”), anterior lumbar interbody fusion (“ALIF”), and transforaminal lumbar interbody fusion (“TLIF”). As the names of these procedures suggest, they are for the lumbar spine, and dependent on the access path into the spine, such as from the posterior side, anterior side, or the lateral side, as well as others. There are also procedures for the cervical and other regions in the spine.

A variety of graft materials are available to promote spinal fusion in between vertebrae, including bone and non-bone graft materials. Bone graft typically includes crushed bone or a combination of crushed bone and other materials, such as synthetic biocompatible materials. The bone graft is meant to stimulate growth of healthy bone, fusing vertebral structures, and improving the stability of the spine. As used herein, bone graft means materials made-up entirely of natural materials, entirely of synthetic biocompatible materials, or any combination of these. These materials may include growth factors that promote bone formation, such as bone morphogenic proteins. Furthermore, there is a range of allograft and autograft materials, with different viscous and fibrous characteristics, handling characteristics, and abilities to be delivered to the implantation site.

Implants for insertion into the spine are known. Surgically implantable intervertebral fusion cages, for example, have been used to perform spinal fusion procedures. Intervertebral fusion cages having fenestrations, capable of being implanted into a patient, such as through an anterior or posterior approach, have been used for spinal surgery. Fenestrated, hollow structures that fit between two vertebrae have been used. Furthermore, insertion instruments for inserting intervertebral implants are known.

In a known method of using an implant and bone graft system, a fusion cage attached to an instrument is inserted into the spine. Bone graft, or other material, is then introduced through a tube around the implant. In another known method, bone graft material is delivered to the surgical site first, leaving an opening for insertion of a fusion cage. Then the fusion cage is inserted, and then the area around the fusion cage is further filled with additional bone graft material. In yet another known procedure, an implant is mounted on an inserter and inserted into the spine, and then graft material is delivered through the inserter, and through the implant, thus surrounding and encasing the implant in the graft material.

Challenges in performing these procedures include streamlining the implant delivery process, facilitating delivery of a wide variety of graft materials, enabling consistent and smooth graft delivery while avoiding jamming and poor graft distribution, and ultimately filling the implant and disk space uniformly, in order to maximize vertebral endplate to endplate contact with graft. Furthermore, reducing the number of instrument passes to the implantation site, reducing the risk of nerve irritation and damage thereby, and reducing the risk of bacterial contamination, are also challenges. Moreover, in addition to offering a static interbody implant, there is a need for offering an expandable interbody implant that can also address all of the points noted above.

As such, there is a need for improved spinal implants, systems and instruments.

### SUMMARY

In an aspect, an implant for being disposed in the spine of a patient comprises an upper plate, and a lower plate, at least one of the upper plate and lower plate having a shaped interior surface facing a wedge piece. The wedge piece is disposed between the upper plate and the lower plate and has a shaped surface engaging the shaped surface of the at least one of the upper plate and lower plate. The implant includes an interface having a driver movable to drive movement of the wedge piece, the shaped surfaces of the wedge piece and the at least one of the upper plate and lower plate sliding against each other to drive movement of the at least one of the upper plate and lower plate.

In embodiments, the shaped surface of the at least one of the upper plate and lower plate is a first shaped surface on an interior surface of the upper plate. A second shaped surface may be on an interior surface of the lower plate.

In embodiments, the shaped surface of the wedge piece is a first shaped surface on an upper side of the wedge piece, the first shaped surface on the upper side of the wedge piece engaging the first shaped surface on the interior surface of the upper plate. The implant can have a second shaped surface on a lower side of the wedge piece, the second shaped surface on the lower side of the wedge piece engaging the second shaped surface on the interior surface of the lower plate. The first shaped surface on the interior surface of the upper plate may be a curved surface. The second shaped surface on the interior surface of the lower plate may be a curved surface.

The first shaped surface on the upper side of the wedge piece and the second shaped surface on the lower side of the wedge piece can be curved surfaces. In embodiments, the first shaped surface and the second shaped surface are disposed on a front end of the wedge piece.

In embodiments, the shaped surface of the wedge piece is disposed on a rear end of the wedge piece. In embodiments, another shaped surface is on a front end of the wedge piece. The shaped surface on the front end of the wedge piece and the shaped surface on the rear end of the wedge piece can be curved. The shaped surface on the front end of the wedge piece can be defined by a first arc and the shaped surface on the rear end of the wedge piece can be defined by a second arc, and the first arc can be concentric with the second arc.

In embodiments, the interface is a threaded interface.

In embodiments, at least one of the upper plate and lower plate have an opening. At least one of the upper plate and lower plate can have textured surfaces on an outer surface.

In embodiments, the implant has an unexpanded condition and an expanded condition, and a first height in the unexpanded condition. The implant can have a second height in the expanded condition. A length of the implant can be greater in the unexpanded condition than a length in the expanded condition. The implant may define a lordosis angle in the expanded condition.

In a further aspect, a system for disposing an implant in a spine of a patient comprises an implant having an upper plate, and a lower plate, at least one of the upper plate and lower plate having a shaped interior surface facing a wedge piece. The wedge piece is disposed between the upper plate and the lower plate and has a shaped surface engaging the shaped surface of the at least one of the upper plate and lower plate. The implant includes an interface having a driver movable to drive movement of the wedge piece, the shaped surfaces of the wedge piece and the at least one of the upper plate and lower plate sliding against each other to drive movement of the at least one of the upper plate and lower plate. The system includes a medical material delivery tube, a plunger receivable in the delivery tube, wherein the implant is removably attachable to the delivery tube and an interior cross-sectional shape of the delivery tube is selected from an oval and a rectangle.

In embodiments, the system has a member for engaging the interface of the implant.

In embodiments, the interface is a threaded interface.

The system can have a funnel removably attachable to a proximal end of the delivery tube.

In another aspect, an implant for being disposed in a spine of a patient comprises an upper plate, and a lower plate, where at least one of the upper plate and lower plate having a first shaped interior surface and a second shaped surface facing a wedge piece. The wedge piece is disposed between the upper plate and the lower plate. The wedge piece has a first shaped surface engaging the first shaped surface of the at least one of the upper plate and lower plate. The first shaped surface is disposed at a front end of the wedge piece, and the wedge piece has a second shaped surface engaging the second shaped surface of the at least one of the upper plate and lower plate. The second shaped surface is disposed at a rear end of the wedge piece. The first shaped surfaces being defined by a first arc, the second shaped surfaces being defined by a second arc, and the first arc and second arc being concentric with one another. The implant has an interface including a driver movable to drive movement of the wedge piece, the shaped surfaces sliding against each other to drive movement of the at least one of the upper plate and lower plate.

In embodiments, the first shaped surface is on the upper plate and the implant further comprises a third shaped surface on an interior surface of the lower plate.

In embodiments, the interface comprises a driver with a front end and a rear end, and the rear end includes sloped surfaced. The wedge piece can have sloped surfaces generally aligned with the sloped surfaces on the driver.

The front end of the driver can be a threaded member. The wedge piece can have an internally threaded hole engaged with the threaded member.

In another aspect, an implant for being disposed in a spine of a patient comprises an upper plate, a lower plate, and a wedge piece disposed between the upper plate and lower plate, the upper plate, the lower plate, and the wedge piece having shaped surfaces that drives expansion of the implant when the wedge piece moves. In embodiments, the wedge piece is moved toward a front end of the implant, expanding the implant, and moving the upper plate and the lower plate away from each other. In embodiments, the implant has a greater height in the expanded condition than a height in the unexpanded condition.

In a further aspect, an implant for being disposed in a spine of a patient comprises an upper plate, a lower plate, a wedge piece disposed between the upper plate and lower plate, and a driver engaged with the wedge piece, the upper plate, the lower plate, and the wedge piece having shaped surfaces that drives expansion of the implant when the wedge piece moves. The driver is moved to move the wedge piece. In embodiments, the wedge piece is moved toward a front end of the implant, expanding the implant, and moving the upper plate and the lower plate away from each other. In embodiments, the implant has a greater height in the expanded condition than a height in the unexpanded condition. In embodiments, the driver and wedge piece are threadably engaged. In embodiments, rotation of the driver moves the wedge piece.

In a further aspect, an implant for being disposed in a spine of a patient comprises an upper plate, a lower plate, a wedge piece disposed between the upper plate and lower plate, and a driver engaged with the wedge piece, the upper plate, the lower plate, and the wedge piece having shaped surfaces that drives expansion of the implant when the wedge piece moves. The driver is moved to move the wedge piece. The driver has sloped surfaces for directing material toward a side of the implant. In embodiments, the wedge piece is moved toward a front end of the implant, expanding the implant, and moving the upper plate and the lower plate away from each other. In embodiments, the implant has a greater height in the expanded condition than a height in the unexpanded condition. In embodiments, the driver and wedge piece are threadably engaged. In embodiments, rotation of the driver moves the wedge piece. In embodiments, the wedge piece has sloped surfaces that may align with the sloped surfaces on the driver for directing material toward a side of the implant.

### BRIEF DESCRIPTION OF THE DRAWINGS

The accompanying drawings, which are incorporated in and constitute a part of this specification, illustrate embodiments and together with the disclosure herein, including the detailed description of the drawings given below, serve to explain principles of this disclosure.

FIG. 1 is a top view of an implant in accordance with embodiments, shown in an unexpanded condition;

FIG. 2 is a side view of an implant in accordance with embodiments, shown in an unexpanded condition;

FIG. 3 is a top view of an implant in accordance with embodiments, shown in an expanded condition;

FIG. 4 is a side view of an implant in accordance with embodiments, shown in an expanded condition;

FIG. 5 is a schematic of concentric circles showing an implant in embodiments having curved surfaces;

FIG. 6 is a schematic of an implant in embodiments having curved surfaces;

FIG. 7 is a chart showing an implant in embodiments in a variety of sizes and configurations; and

FIG. 8 is a plan view of a kit in accordance with embodiments.

### DETAILED DESCRIPTION OF ILLUSTRATIVE EMBODIMENTS

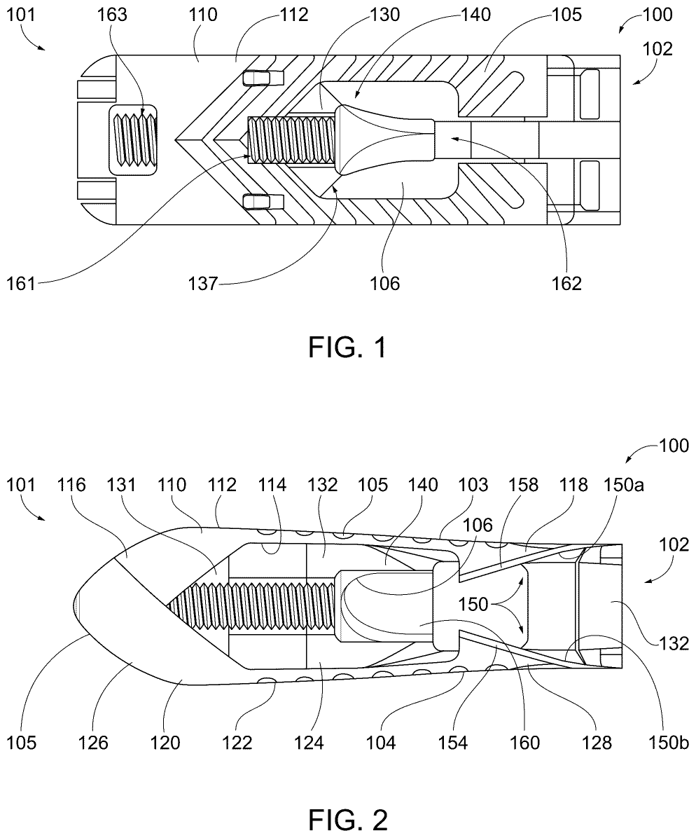

An embodiment of a spinal implant is shown in FIGS. 1-6. This implant is intended to be used in an intervertebral fusion procedure. In embodiments, an expandable intervertebral implant 100 includes an upper plate 110, a lower plate 120, an inner wedge piece 130, and an interface 140. The wedge piece is disposed between the upper plate and the lower plate and is movable with respect to the upper plate and the lower plate. The implant 100 is delivered on an inserter instrument, which will be discussed further below, and includes a tube. The implant 100 has a front end 101, a rear end 102, a top side 103, and a bottom side 104. The wedge piece 130 has a front end 131 and a rear end 132.

In embodiments, the upper plate 110 and lower plate 120 include curved outer surfaces 105 at the front end 101 of the implant. The upper plate 110 has an outer surface 112, an inner surface 114, a front end 116, and a rear end 118. The lower plate 120 has an outer surface 122, an inner surface 124, a front end 126, and a rear end 128. The upper plate 110 inner surface 114 and lower plate 120 inner surface 124 have a curved surface at their respective front ends 116 and 126. For example, the upper plate and lower plate include inner surfaces at the front end 101 of the implant that each have a curved arc. The upper plate front inner curved surface is shown at 115 and the lower plate front inner curved surface is shown at 125. In embodiments, these surfaces correspond with, and interact with, shaped surfaces 132 and 134 on the front end 131 of the wedge piece 130.

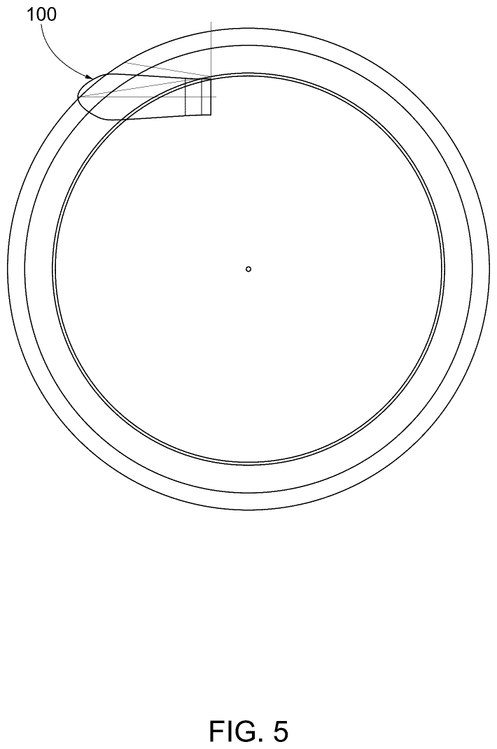

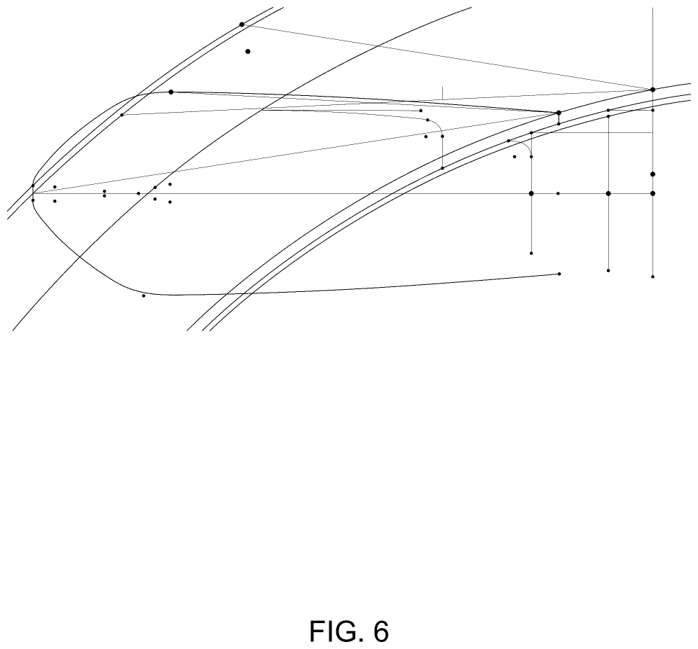

A rear end 102 of the implant 100, in embodiments, includes curved surfaces 150 facing the upper plate 110 and lower late 120. In embodiments, the upper plate 110 and lower plate 120 have inner rear end surfaces for interacting with the surfaces 150. For example, the upper plate 110, at the rear end 118, includes a curved surface 152, and the lower plate 120, at the rear end 128, also includes a curved surface 154. The curved surfaces 152 and 154 may be arc shaped. FIGS. 5 and 6 show the shape of the curved surfaces at the front end 101 and rear end 102 of the implant 100, and their concentric relationship with one another. In embodiments, the implant 100 has shaped surfaces at the front end, shaped surfaces at the rear end, or both. These surfaces are curved. However, other shapes are contemplated, such as wedge-shaped ramps.

In embodiments, the implant has curved surfaces on the wedge piece and curved surfaces on the upper plate and lower plate that interact when the wedge piece is moved. This drives movement of the upper plate and the lower plate in vertical directions, away from each other, moving the implant to an expanded condition. In embodiments, the curved surfaces lie on concentric circles. This is illustrated in FIGS. 5 and 6.

The center point of the concentric circles and the radius of the circles, as well as the sizes of the implant, itself, can be varied to arrive at implants 100 of different sizes and shapes. For example, the circles can be varied in this way to provide an implant with a greater height, and a lesser or greater lordosis angle. For another example, the circles can be varied to provide an implant with a greater lordosis angle, and a lesser or greater height.

In embodiments, the upper plate 110 and lower plate 120 have sloped surfaces on their inner surfaces at the front end, sloped surfaces on the inner surfaces at their rear ends, curved surfaces, or some combination thereof. The surfaces on the wedge piece 130 are shaped to correspond to the surfaces on the upper plate 110 and lower plate 120 and interact with the surfaces on the upper plate and lower plate as discussed further below.

The inner surfaces 114 and 124 of the upper plate 110 and the lower plate 120 define a space for the wedge piece 130. The wedge piece 130 has a hole 136 having a shape for interfacing with a driver 160. For example, the hole 136 may be threaded and interact with a screw type threaded member for driving movement of the wedge piece 130. In embodiments, the driver 160 has a slot that engages a ridge on the wedge piece 130. Other inter-engaging features are contemplated, such as hexagonal or other polygonal shapes in female and male members. In embodiments, operation of the driver 160 moves the wedge piece 130 towards the front end 101 of the implant 100, or away from the front end 101 of the implant. For example, the interface 140 between the driver 160 and the hole 136 of the wedge piece 130 can be a threaded arrangement. However, other interfaces are envisioned. In embodiments, rotation of the driver 160 moves the wedge piece 130 toward the front end 101 of the implant 100.

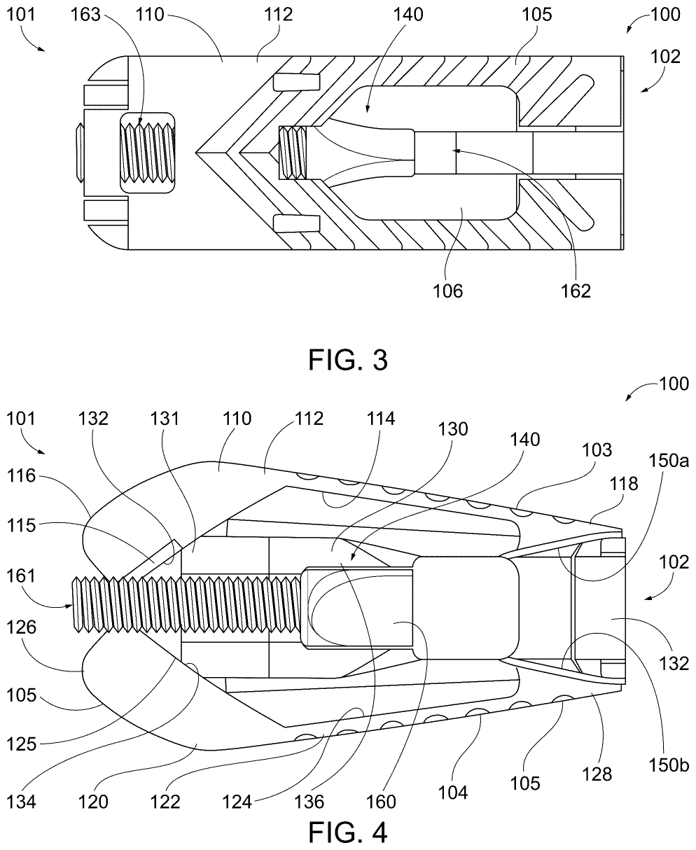

In embodiments, the driver 160 is engaged by a member 162. The member 162 may be integrated with the insertion instrument, or the member 162 may be inserted down the interior of the inserter instrument. The member 162 can have, for example, a hexagonal or similar polygonal shaped member that engages the driver 160. For example, the driver can have an aperture having a shape for engaging the member. In this way, the member is used to drive the operation of the wedge piece 130. In embodiments, the driver 160 has a ridge and the member 162 has a slot that engages the ridge so that when the member 162 is rotated, the driver 160 and screw rotates (see FIG. 3).

The wedge piece 130 has at its front end 131 curved outer surfaces 132 and 134 on an upper side and a lower side. These curved outer surfaces interact with the curved inner surfaces at the front end 116 of the upper plate 110 and front end 126 of the lower plate 120. In embodiments, these curved surfaces have the shape of an arc. In embodiments, the implant 100 also has rear curved surfaces 150 that interact with the inner curved surfaces at the rear end 118 of the upper plate 110 and rear end 128 of the lower plate 120. In embodiments, the rear curved surfaces 150 of the implant 100 are provided on the wedge piece 130 at surfaces 150a and 150b. In embodiments, the curved surfaces are concentric (see FIGS. 5 and 6), leading to expansion of the implant 100 in a smooth transition. In embodiments, the shape of the expanded implant 100 follows the shape of the lordosis angle of the vertebral bodies of the spine. In embodiments, the expanded implant 100 defines a lordosis angle. In embodiments, there is a continuous height and lordotic change as the wedge piece 130 is moved, due to the shape of the curved surfaces. In embodiments, the interface 140 between the wedge piece 130 and the driver 160 moves the wedge piece 130 toward the front end 101 of the implant 100, driving the upper plate 110 and the lower plate 120 away from the wedge piece 130 in vertical directions, through the interaction of the curved surfaces. In embodiments, the rear curved surfaces of the implant interact with inner curved surfaces on the upper plate and lower plate, providing a smooth transition between the unexpanded and expanded condition of the implant. In embodiments, the expansion is adjustable to provide for a variety of different heights in the expanded implant. In embodiments, one or more of the inner surfaces of the upper plate 110 and lower plate 120 are engaged with the surfaces of the wedge piece 130 and/or implant 100 along a track. For example, these surfaces may have a dovetail-shaped interface guiding the movement of the upper plate 110 and lower plate 120.

In embodiments, the implant 100 has interior surfaces that direct bone graft material out the sides of the implant. In embodiments, the front end of the implant is obstructed, for example, such as by surfaces of the upper plate and lower plate. In this way, bone graft material is directed out the sides of the implant and not out the front end of the implant. In embodiments, the driver 160 comprises a bolt having a threaded distal end near the front end 101 of the implant. The driver 160 has a proximal end, for example, with an outwardly facing surface 163 that is curved or angled towards the sides of the implant for directing the bone graft material out the sides of the implant 100 (see FIG. 1). In embodiments, the wedge piece 130 has curved surfaces 137 that align with the curved surfaces on the driver 160 (see FIG. 1), to further assist in encouraging bone graft material to flow towards the sides of the implant 100 and out the sides of the implant 100.

In embodiments, the driver 160 interacts with a detent that provides a tactical indication of the orientation of the curved surfaces on the driver 160. In this way, the surgeon will know when the driver 160 curved surfaces are aligned with the curved surfaces 137 on the wedge piece 130, so that the bone grafting flow guiding aspect of the interface 140 is maximized. This detent feature, for example, also assists the surgeon in maintaining the position of the driver 160 temporarily. If desired, the surgeon can overcome the detent to further rotate the driver 160, reverse the rotation of the driver, in certain embodiments.

In embodiments, the upper plate and lower plate outer surfaces have a textured surface 105. The textured surface can comprise striated surfaces. The upper plate and lower plate in embodiments include one or more openings 106 that face the adjacent vertebrae.

The system may be modular in that components may be offered in various sizes and configurations. The upper plate and lower plate of the implant can be provided in several sizes. In embodiments, the upper plate and lower plate are identical. However, different configurations for the upper plate and the lower plate are contemplated. The wedge piece can be provided in assorted sizes. The upper plate, lower plate, and wedge piece can be provided in different combinations. In embodiments, the upper plate, lower plate and wedge piece are provided so that the overall implant has a height of 7 mm in its unexpanded state. In another example, the upper plate, lower plate, and wedge piece are provided so that the implant has a height of 9 mm in its unexpanded state. In this example, the 9 mm size achieves a higher lordosis height than the 7 mm size. In embodiments, the components are arranged to define different lordosis angles.

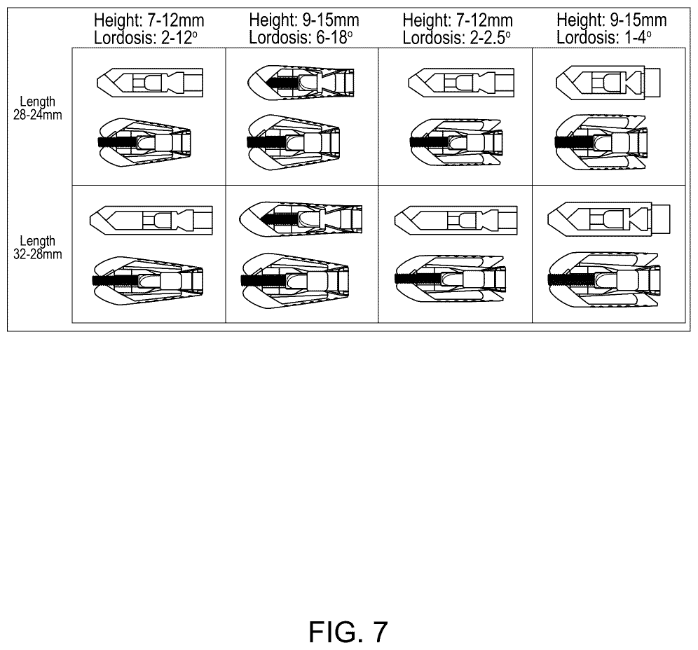

In embodiments, implants of the types described herein are provided in a variety of sizes and configurations. FIG. 7 illustrates a system including intervertebral implants in a variety of sizes and configurations. For example, in a modular system, where components may be combined and various instruments provided in the system, several sizes and configurations for the implant are included. The implant may vary in length, for example, from about 24 millimeters (“mm”) to 36 mm, generally. In embodiments, the lengths of the implants can be, in a first size of 28 mm and, in a second size of 32 millimeters, in the initial unexpanded configuration. In embodiments, the 28 mm length implant has a length of 24 millimeters in the expanded configuration. In embodiments, the 32 mm length implant has a length of 28 mm in the expanded configuration.

The implants may have a height from 7 mm to 12 mm, generally. In embodiments, an implant in a first size has an initial height of 7 mm and a height of 12 mm in the expanded configuration. In another size, the implant has a height of 9 mm initially and a height of 15 millimeters in the expanded configuration. The lordosis angles can vary from 2° to 20°, generally. In embodiments, implants are provided with lordosis angles of 12 degrees in one size, 18° in another size, 2.5° in a further size, and 4° in another size, in the expanded configuration. FIG. 7 illustrates a specific example of a system including implants in eight different configurations, as shown.

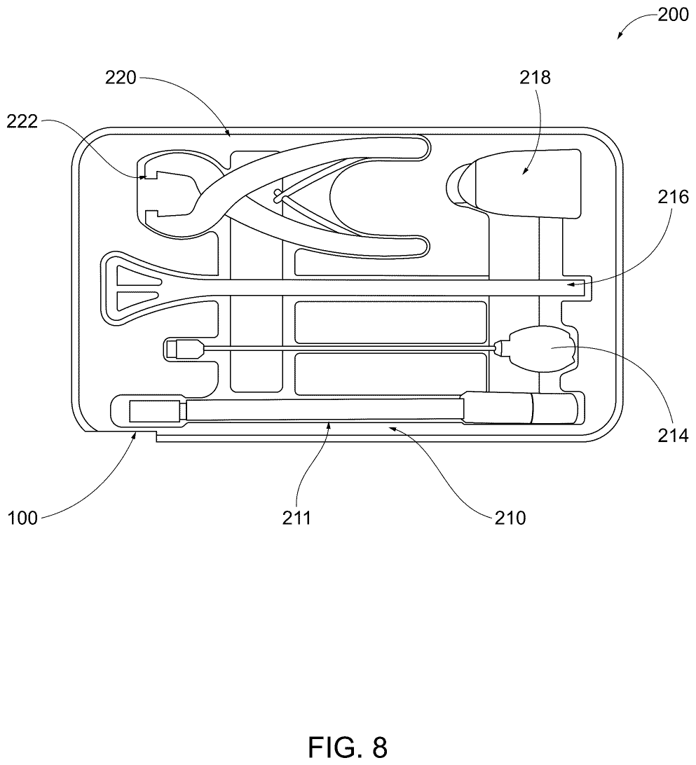

An embodiment of a surgical kit 200 is shown in FIG. 8. The kit has an instrument inserter 210. In embodiments, the inserter 210 has a spinal implant attached to the distal end of the inserter instrument 210. In embodiments, the implant 100 has a large opening at the rear end 102 where the inserter instrument attaches. The spinal implant 100 and instrument inserter 210 can be provided already assembled in the kit 200. The kit 200, in embodiments, has an actuator 214 that is used to engage the implant 100 and expand the implant. The kit can also include, in embodiments, a funnel 218 that is shaped so as to be removably attachable to the proximal end of the inserter instrument 210. The kit 200 can include removal pliers 220. In embodiments, the removal pliers 220 have protrusions 222. When the removal pliers 220 engage the proximal end of the inserter instrument 210, the implant 100 becomes detached from the inserter instrument 210.

The kit 200 in embodiments, comes with the implant pre-assembled to the hollow tube 211 of the inserter instrument 210. In embodiments, the implant 100 is the expandable implant 100 discussed above. In embodiments, the implant can have different configurations.

In embodiments, the inserter instrument 210 has an actuator 214 with the member 162 for engaging the driver 160 of the expandable implant 100. Rotation of the driver 160 rotates the expansion screw 161. The implant 100 is expanded by the inserter instrument 210 as a result of the driver engaging the expansion screw 161 and rotating, expanding the implant, while the tube 211 of the instrument inserter 210 is engaged with the implant 100.

After the implant is expanded to the desired height, the actuator 214 disengages from the driver 160 and is removed. The hollow tube 211 of the instrument inserter 210 is then used to deliver bone graft material. This occurs while the implant 100 is attached to the instrument inserter 210. For example, the tube 211 is an integral part of the inserter instrument. Thus, no additional steps of introducing a bone graft delivery system are required. The tube 211 is used to deliver bone graft material into the implant 211 and to the surrounding surgical site through the implant 100. In embodiments, the implant 100 is removably engaged with the hollow tube 211.

After the implant has been positioned in the surgical site and expanded, bone graft material is inserted into the inserter instrument 210, advanced down the tube 211, into the implant 100 and into the surrounding surgical site. Bone graft material is placed into the tube 211, such as for example, through the funnel 218, or through a syringe, and advanced down the hollow tube 211 by the plunger 216. Bone graft material flows into the implant 100 and into the surgical site. For example, the implant 100 may have a closed distal end and open sides whereby the bone graft material flows through the implant and outward into the surgical site.

The inserter, bone graft delivery tube, and integrated fusion cage with expandable implant plates, is designed to receive bone graft material at the proximal end so that the material flows through the tube, into the implant, and into the surgical site. In embodiments, the implant has interior ramps which function to direct bone graft material to the surgical site. The features of the hollow tube and plunger, in embodiments, are sized and shaped to deliver a greater volume of bone graft material, while reducing blockages or jamming. In embodiments, the tube and plunger are sized and shaped to interact to deliver a greater volume of bone graft material to the surgical site. The cross-sectional surface area of the graft cannula at its junction with the implant does not change or decrease; this allows the system to be devoid of pinch points and reduces the risk of jamming.

In embodiments, an installer instrument 210 has features for detaching the implant 100. The system 200 can include removal pliers 220 configured to engage the proximal end of the hollow tube and installer instrument 210. The pliers 220, for example, engage the proximal end of the installer instrument 210 and engage features that disengage the implant 100 from the inserter instrument 210. In embodiments, the installer 210 has a tip with movable parts that spread to disengage the implant. For example, the implant 100 may have a detent, such as for example, on a proximal part having a groove. For example, the installer tip expands to disengage from the groove thereby detaching the implant. This is actuated by the engagement of the pliers 220 with the proximal end of the installer instrument 210.

In embodiments, the inserter instrument 210, near the proximal end of the tube 211, has an indicator to indicate to the surgeon the state of the expansion of the implant 100. For example, the indicator can indicate the number of turns of the member 162 and driver 160, the height of the implant 100 as the implant 100 is being expanded, the orientation of the sloped surfaces on the driver 160, the length of the implant 100 during expansion, or other information.

In embodiments, the tube 211 of the instrument 210 has an oblong shape in cross section. The tube 211 has a passageway for delivering bone graft material to a distal end of the tube. The passageway, for example, can have an oblong, or non-round cross-sectional shape. For example, the tube can be rectangular or oval in cross-sectional shape. The system 200 includes a plunger 216 with a shape corresponding to the bone graft delivery tube 211. The plunger 216 is used to convey bone graft material to a distal end of the tube 211.

The plunger 216, in embodiments, comprises a handle and a proximal end and a plunger distal end. In embodiments, the plunger 216 is generally symmetrical in shape and corresponds to the inner cross-sectional shape of the tube 211. In embodiments, the plunger forms a congruent or conformal engagement with the interior of the hollow tube 211.

What is claimed is:

1. An implant for being disposed in the spine of a patient, comprising:
an upper plate;
a lower plate, at least one of the upper plate and lower plate having a shaped interior surface facing a wedge piece,
the wedge piece being disposed between the upper plate and the lower plate and having a shaped surface engaging the shaped surface of the at least one of the upper plate and lower plate; and
an interface defined between the wedge piece and the at least one of the upper plate and lower plate, the interface including a driver movable to drive movement of the wedge piece, the shaped surfaces of the wedge piece and the at least one of the upper plate and lower plate sliding against each other to drive movement of the at least one of the upper plate and lower plate.

2. The implant of claim 1, wherein the shaped surface of the at least one of the upper plate and lower plate is a first shaped surface on an interior surface of the upper plate.

3. The implant of claim 2, further comprising a second shaped surface on an interior surface of the lower plate.

4. The implant of claim 3, wherein the shaped surface of the wedge piece is a first shaped surface on an upper side of the wedge piece, the first shaped surface on the upper side of the wedge piece engaging the first shaped surface on the interior surface of the upper plate.

5. The implant of claim 4, further comprising a second shaped surface on a lower side of the wedge piece, the second shaped surface on the lower side of the wedge piece engaging the second shaped surface on the interior surface of the lower plate.

6. The implant of claim 5, wherein the first shaped surface on the interior surface of the upper plate is a curved surface.

7. The implant of claim 6, wherein the second shaped surface on the interior surface of the lower plate is a curved surface.

8. The implant of claim 7, wherein the first shaped surface on the upper side of the wedge piece and the second shaped surface on the lower side of the wedge piece are curved surfaces.

9. The implant of claim 8, wherein the first shaped surface and the second shaped surface are disposed on a front end of the wedge piece.

10. The implant of claim 1, wherein the shaped surface of the wedge piece is disposed on a rear end of the wedge piece.

11. The implant of claim 10, further comprising another shaped surface on a front end of the wedge piece.

12. The implant of claim 11, wherein the shaped surface on the front end of the wedge piece and the shaped surface on the rear end of the wedge piece are curved.

13. The implant of claim 12, wherein the shaped surface on the front end of the wedge piece is defined by a first arc and the shaped surface on the rear end of the wedge piece is defined by a second arc, the first arc being concentric with the second arc.

14. The implant of claim 1, wherein the interface is a threaded interface.

15. The implant of claim 1, wherein at least one of the upper plate and lower plate have an opening.

16. The implant of claim 1, wherein at least one of the upper plate and lower plate have textured surfaces on an outer surface.

17. The implant of claim 1, wherein the implant has an unexpanded condition and an expanded condition, and a first height in the unexpanded condition.

18. The implant of claim 17, wherein the implant has a second height in the expanded condition.

19. The implant of claim 18, wherein a length of the implant is greater in the unexpanded condition than a length in the expanded condition.

20. The implant of claim 17, wherein the implant defines a lordosis angle in the expanded condition.
